(19) United States
(12) Patent Application Publication
(10) Pub. No.: US 2025/0326792 A1
(43) Pub. Date: Oct. 23, 2025

(54) A FORMULATION FOR TREATMENT OF PIGMENTARY DISORDERS

(71) Applicant: Ramakrishna Reddy ISANAKA, Hyderabad, Telangana (IN)

(72) Inventor: Ramakrishna Reddy ISANAKA, Hyderabad, Telangana (IN)

(21) Appl. No.: 18/547,294

(22) PCT Filed: Oct. 19, 2022
(86) PCT No.: PCT/IB2022/060046

§ 371 Date: Aug. 21, 2023

(30) Foreign Application Priority Data
Jan. 5, 2022 (IN) 202241000559

(51) Int. Cl. C07K 7/06; A61K 38/00; A61P 17/00

(52) U.S. Cl. CPC C07K 7/06; A61P 17/00; A61K 38/00

(57) ABSTRACT

The present invention relates to a peptide for treatment of pigmentary disorders. The invention also relates to stable topical formulation for treatment of pigmentary disorders. The present invention further relates to method of preparation of such formulation and uses thereof for treatment or prevention of pigmentary disorders.

### CROSS REFERENCE TO RELATED APPLICATIONS

This application is a National Stage application of PCT/IB2022/060046, filed Oct. 19, 2022, which claims priority to Indian Application No. 202241000559, filed Jan. 5, 2022, and all the benefits accruing therefrom under 35 U.S.C. § 119, the content of which is incorporated by reference in its entirety.

### SEQUENCE LISTING

The Instant Application contains a Sequence Listing which has been submitted electronically in XML format and is hereby incorporated by reference in its entirety. Said XML copy, created on Apr. 11, 2024, is named “SMN0016US” and is 2,180 bytes in size. The Sequence Listing does not go beyond the disclosure in the application as filed.

### FIELD OF THE INVENTION

The present invention relates to a peptide for treatment of pigmentary disorders. The invention also relates to stable topical formulation comprising said peptide. The present invention further relates to method of preparation of such formulation and uses thereof for treatment, prevention and/or amelioration of one or more symptoms of pigmentary disorders.

### BACKGROUND OF THE INVENTION

Skin colour depends on the synthesis and distribution of pigment melanin by specialized skin cell melanocytes, which along with adjacent keratinocytes constitute epidermal melanin unit [1-4]. Melanin is a natural pigment synthesized and stored in specialized organelles, termed melanosomes, in melanocytes [5-6]. In skin and hair, the melanosomes are transported to dendrites of melanocytes and then transferred to the neighbouring keratinocytes and provides protection to the skin and the body against deleterious effect of UV irradiation. Biosynthesis of melanin is a very complex phenomenon and controlled at various points. The synthesis and expression of different melanogenic enzymes and their inhibitors play a crucial role in accomplishing melanin synthesis inside melanosome. Melanogenesis is the process of melanin synthesis in melanocytes. Two types of melanin are present in mammals: a black/brown eumelanin and a red/yellow pheomelanin. Ratio of eumelanin and pheomelanin determines diversity of skin and hair pigmentation in humans. Synthesis of both types of melanin begins with amino acid L-tyrosine. Tyrosinase, a copper containing key regulatory bifunctional enzyme in melanin biosynthesis, catalysis conversion of essential amino acid L-tyrosine to L-3, 4-dihydroxyphenylalanine (L-DOPA), the rate-limiting step, for both eumelanin and pheomelanin. Eumelanin synthesis additionally requires enzymes tyrosinase-related protein 2 (Trp2) and Trp1, whereas pheomelanin additionally requires amino acid cysteine [7]. A well-known factor that can induce tyrosinase expression is α-melanocyte stimulating hormone (α-MSH), which binds melanocortin 1 receptor (MC1R) to activate adenylyl cyclase to produce cAMP. The CAMP activates cAMP-dependent kinase A (PKA) and increases the expression of melanocyte-specific microphthalmia-associated transcription factor (MITF), a master regulator for expression of the melanogenic enzymes tyrosinase, Trp1 and Trp2. Induction of melanogenesis in the skin is mainly influenced and controlled by locally released peptide like stimulators or hormones such as ET-1, ACTH, α-MSH and β-endorphin. They impart such action by enhancing tyrosinase and other melanogenic enzyme activity and expression through receptor mediated mechanism [8-15].

Insufficient synthesis of melanin leads to several human hypopigmentation disorders, such as vitiligo, pityriasis alba, tinea versicolor, post inflammatory hypopigmentation under category acquired (common) hypopigmentation disorders; and albinism, piebaldism, tuberous sclerosis, hypo melanosis of Ito under category congenital (uncommon) hypopigmentation disorders. Commonly known hypopigmentation disorders are hair greying and vitiligo [16]. A recent study showed that 74% of people between 45 and 65 years of age have grey hair [17]. Vitiligo is a frequent cause of depigmentation worldwide with an estimated prevalence of 1% [18]. There are several treatments for hypopigmentation disorders, the efficacy and safety remain unsatisfied and need to be improved [19]. The known treatments are associated with potential mutagenic and adverse effects including skin irritation, contact dermatitis, low stability toward oxygen, high toxicity and insufficient skin penetration ability and water, and exogenous chronosis.

EP1030914A2 discloses specific, optionally modified oligonucleotides with a length of up to 18 nucleotides, preferably with a length of 7-15 nucleotides, which correspond to sections of tenascin-coding sequences and which can bind to these sequences, their preparation and their use, for example for specific inhibition of the expression of tenascin and for the manufacture of medicinal products which can be used for the treatment of vitiligo.

U.S. Pat. No. 7,087,743B2 discloses oligonucleotides and use of oligonucleotides modulating the expression of enzymes involved in the synthesis of melanic pigments, as depigmentation agents. The disclosed oligonucleotide sequences are capable of hybridizing specifically with the gene or with a product of the gene encoding tyrosinase, or with the gene or a product of the gene encoding tyrosinase-related-protein 1 (TRP-1).

WO2018012889A1 discloses a composition for prevention or treatment of vitiligo or vitiligo metastasis comprising an interferon-inducible T-cell alpha chemoattractant inhibitor.

U.S. Pat. No. 8,314,065B2 discloses agonist peptides of basic fibroblast growth factor (bFGF) and the method of reduction of wrinkles on skin, darkening of hair, acceleration of wound healing and synergistic therapies for the treatment of vitiligo.

WO2019142124 discloses a topical pharmaceutical composition comprising a therapeutically effective amount of the bFGF and at least one additional therapeutic agent, along with at least one suitable pharmaceutically acceptable carrier, diluents, vehicle or excipient. WO2007032029 discloses a composition containing agonist peptides of bFGF in combination with any known acceptable carrier for topical application for reduction of wrinkles on skin, in acceleration of wound healing and darkening of hair. Current drug therapies have side effects such as being toxic to liver, carcinogenic, and are not recommended to paediatrics owing to long term side effects. The current therapies are expensive therefore not affordable for long term use and produce undesirable effects upon usage.

An imbalance in cytokine expression is observed at vitiliginous lesions level, primarily on the border of lesional and perilesional patches with non-lesional skin areas. The increase of pro-inflammatory cytokines has probably originated by a shift of the immune response, characterized by the prevalence of T helper Th1/Th17-related cytokines (pro-inflammatory) instead of a Tregs/Th2-related one (anti-inflammatory) and may be part of the etiology of this autoimmune disease. Another important pro-inflammatory mediator TNF-α plays a pivotal role in oxidative stress-enhanced cytotoxicity against both melanocytes and keratinocytes. The fundamental role of a chronic inflammatory status clearly appears in vitiligo etiopathology; the impaired crosstalk between two chief skin cellular subsets (keratinocytes and melanocytes) due to excessive oxidative stress and an altered immune response in vitiliginous areas are both driven by the loss of the homeostatic levels of involved specific cytokines and growth factors. The comprehension of intra- and intercellular signalling pathways is crucial to better understand the vitiligo pathological processes and to study innovative and effective therapeutic approaches. These effects are probably due to cytotoxic T lymphocytes, which produce pro-inflammatory cytokines such as TNF-α and IFN-γ. These pro-inflammatory cytokines sensitize keratinocytes to undergo apoptosis highlighting the role of inflammation in vitiligo onset and spreading. Vitiligo is characterized by alteration of the immunological balance, primarily reflected in an imbalance between the cytokines expressed by Th1/Th17 (TNF-α; IFN-γ; IL-1; IL-17; IL-2; IL-6; IL-8) and by Treg/Th2 lymphocyte subsets (IL-4). Th1 cytokines hyper-production is linked with autoimmune diseases and vitiligo fully fits with this pathological immune picture, characterized by an important inflammatory component.

bFGF is a potent mitogen for a variety of cell types including melanocytes. bFGF or its active short peptides are mitogenic to melanocytes and stimulate melanogenesis. Therefore, bFGF derived peptides (bFGFRP) may be effective in re-pigmentation of depigmented patches on skin. Thus, active peptides derived from b-FGF may be potential therapeutic agents for managing pigmentary disorders, particularly hypopigmentation disorders.

Tyrosinase is a crucial enzyme responsible for melanin synthesis. Identifying molecules that can modulate tyrosinase expression and activity is recognized to be crucial for developing agents to treat pigmentation disorders. Hence, identifying molecules that can regulate tyrosinase expression and activity is a worthwhile and constructive approach for developing agents for treating pigmentation disorders.

Melanocortin 1 receptor (MC1R), also known as melanocyte-stimulating hormone receptor (MSHR), melanin-activating peptide receptor, or melanotropin receptor, is a G protein-coupled receptor that binds to a class of pituitary peptide hormones known as melanocortin's, which include adrenocorticotropic hormone (ACTH) and different forms of melanocyte-stimulating hormone (MSH). MC1R is one of the key proteins involved in regulating mammalian skin and hair color. It is located on plasma membrane of melanocytes which produce pigment melanin through process of melanogenesis. MC1R works by controlling type of melanin being produced and its activation causes melanocyte to switch from generating yellow or red pheomelanin to brown or black eumelanin in replacement. MC1R has also been reported to be involved in cancer (independent of skin coloration), developmental processes, and susceptibility to infections and pain.

New therapeutic options which can contribute to improving the quality of life of patients and advance search for a truly effective treatment of vitiligo are needed.

Despite of all the studies and research till date, development of high-performance tyrosinase inhibitors is much needed. There is a need to develop new and effective agents for pigmentary diseases. Further, there is a need for new topical formulations which can suppress autoimmune targeting of melanocytes and promote melanocyte regeneration. The new topical formulations should have high shelf life by virtue of long-term storage stability.

### DISCLOSURE OF THE INVENTION

The present invention provides IS103, single therapeutic agent, a decapeptide of SEQ. ID NO 1 from bFGF class for treatment, prevention and/or amelioration of one or more symptoms of pigmentary disorders in a formulation. The present invention also provides a stable pharmaceutical formulation of therapeutically effective amount of decapeptide of SEQ. ID NO 1 along with one or more suitable pharmaceutically acceptable agents, suitable carriers, diluents, vehicles, or excipients. The formulation is preferably in a topical form. The present invention provides use of the pharmaceutical formulation for treatment, prevention and/or amelioration of one or more symptoms of pigmentary diseases. The invention also describes method for preparation of such stable formulation. The invention further provides method for treatment, prevention and/or amelioration of one or more symptoms of pigmentary diseases by the stable formulation. The formulation of present invention is suitable for cosmetic preparations meant for treatment, prevention and/or amelioration of one or more symptoms of vitiligo.

In an embodiment, the present invention provides a pharmaceutical formulation comprising 0.01 to 1% w/v of a peptide of SEQ. ID NO 1 or variant thereof and one or more suitable pharmaceutically acceptable excipients for treating, preventing and/or ameliorating one or more symptoms of pigmentary disorders.

In another embodiment, the one or more suitable pharmaceutically acceptable excipients in the formulation are selected from the group consisting of suitable carriers, diluents, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants.

In a yet another embodiment, the present invention provides that the peptide variant in the formulation is at least 80%, at least 85%, at least 90%, at least 95%, at least 97%, at least 99% identical to the SEQ. ID NO 1.

In a yet another embodiment, the present invention provides that the formulation is suitable for topical mode of administration.

In another embodiment the present invention provides that the formulation is in form of a gel, ointment, creams, lotion, solution and foams.

In another embodiment, the present invention provides that the formulation treats or prevents and/or ameliorate one or more symptoms of pigmentary disorders from the group hypopigmentation disorders from the group vitiligo, pityriasis alba, tinea versicolor, albinism, piebaldism, tuberous sclerosis, hypo melanosis of Ito.

In a yet another embodiment, the present invention provides a formulation for prevention and/or delay of progression of vitiligo or for reversal of depigmentation and/or increasing melanin of patients suffering from vitiligo.

In a still another embodiment the formulation treats, prevents and/or ameliorate one or more symptoms of pigmentary disorders such as patchy loss of skin color, premature whitening or graying of hair, loss of color in tissues.

In one of the embodiments, the present invention provides a method of preparing pharmaceutical formulation for treating, preventing and/or ameliorating one or more symptoms of pigmentary disorders comprising the steps:
a) adding required quantity of water and decapeptide of SEQ. ID No 1 or variant thereof in a container and stirring at suitable speed by suitable means until clear solution is obtained;
b) adding required quantity of sucrose is a separate container and stirring at suitable speed by suitable means until completely dissolved;
c) adding sucrose solution obtained in step ‘b’ to clear solution obtained in step ‘a’;
d) adding required quantity of pharmaceutically acceptable excipients to clear solution obtained in step ‘a’ under continuous stirring until a clear homogenous solution of the pharmaceutical formulation is obtained.

In another embodiment the present invention provides that the method of preparing the formulation further comprises packaging of said solution in suitable containers.

In another embodiment the present invention provides that the method of preparing the formulation optionally comprising sterilizing the pharmaceutical formulation by suitable sterilization methods before or after packaging of said pharmaceutical formulation in suitable containers.

In an embodiment the present invention provides that the suitable speed in step ‘a’ is 150 rpm.

In another embodiment the present invention provides that the pharmaceutically acceptable excipients in step ‘d’ comprises isopropyl alcohol, dimethyl sulfoxide and propylene glycol.

In another embodiment the present invention provides that a method of treating, preventing and ameliorating one or more symptoms of pigmentary disorders in a subject in need thereof, comprising administering a therapeutically effective amount of a formulation comprising a therapeutically effective amount of peptide of SEQ. ID NO 1 or variant thereof wherein the formulation enhances melanin synthesis and/or suppresses autoimmune targeting of melanocytes and/or inhibits tyrosinase activity and/or reduces levels of cytokines.

In another embodiment, the present invention provides that in the method of treating, preventing and ameliorating one or more symptoms of pigmentary disorders in a subject in need thereof, the therapeutically effective amount of peptide of SEQ. ID NO 1 or variant thereof in the formulation is 0.01 μg/ml to 10,000 μg/ml.

In another embodiment the present invention provides that in the method of treating, preventing and ameliorating one or more symptoms of pigmentary disorders in a subject in need thereof, the formulation is administered to the subject through topical mode of administration.

In another embodiment, the present invention provides use of the formulation for preparation of a medicament for treating, preventing or reducing the severity of pigmentary disorders in an individual.

In a yet another embodiment, the present invention provides use of the formulation for treating, preventing and/or ameliorating one or more symptoms of pigmentary disorders in a subject in need thereof, comprising administering to the subject a therapeutically effective amount of the formulation, wherein the formulation is capable of altering one or more of melanin synthesis, tyrosinase activity and level of cytokines.

In a yet another embodiment, the present invention provides a method of manufacturing decapeptide IS103 of SEQ. ID NO 1 or variant thereof comprising the steps:
a) synthesizing said decapeptide by coupling one amino acid at a time, starting from C-terminus amino acid which is attached to a solid resin via a linker group;
b) controlling coupling of step a) by varying de-protection time and reagents, wherein de-protection is performed twice;
c) drying and weighing peptide resin obtained after coupling last amino acid;
d) cleaving resin-bound peptide off said resin by trifluoroacetic acid to obtain crude peptide;
e) optionally processing crude peptide obtained in step d) by reverse phase chromatography and ion exchange to obtain solution of purified peptide;
f) optionally lyophilizing said solution of purified peptide for removal of residual solvents.

### DESCRIPTION OF THE DRAWINGS

FIG. 1 demonstrates 3-D structure and chemical structure of decapeptide IS103.

FIG. 2 demonstrates in silico docking scores and interaction pattern of IS103 and α-MSH with MC1R.

FIG. 3 demonstrates mode of action of IS103 on tyrosinase activity. The figure shows that binding of IS103 to MC1R enhances melanin synthesis. MC1R represents Melanocortin 1 receptor, MITF represents Microthalamia-associated transcription factor; TRP-1 represents tyrosinase related protein 1, TRP-2 represents tyrosinase related protein 2, AC represents Adenylyl cyclase-2, PME represents premelanosome protein.

FIG. 4 demonstrates that IS103 enhanced melanin synthesis in a dose-dependent manner in B16F10 cells. B16F10 cells were cultured in 6-well plates and treated with isobutyl methylxanthine (IBMX) or α-MSH or IS103 at different concentrations, as indicated, for about 48 hours. Cell viability was analysed by XTT assay. Data are represented as mean+SD from three independent experiments. p<0.001.

FIG. 5 demonstrates that IS103 enhanced melanin synthesis in a dose-dependent manner in NHEM cells. NHEM cells were cultured in 6-well plates and treated with isobutyl methylxanthine (IBMX) or α-MSH or IS103 at different concentrations, as indicated, for about 48 hours. Cell viability was analysed by XTT assay. Data are represented as mean+SD from three independent experiments. p<0.001.

FIG. 6 demonstrates that IS103 increased tyrosinase activity in B16F10 cells. B16F10 cells were cultured in 6-well plates and treated with isobutyl methylxanthine (IBMX) or α-MSH or IS103 at different concentrations, as indicated, for about 48 hours. The cell lysates were used to perform tyrosinase activity assay. Data are represented as the means+SD from three independent experiments. p<0.001.

FIG. 7 demonstrates that IS103 increased tyrosinase activity in NHEM cells. NHEM cells were cultured in 6-well plates and treated with isobutylmethylxanthine (IBMX) or α-MSH or IS103 at different concentrations, as indicated, for about 48 hours. The cell lysates were used to perform tyrosinase activity assay. Data are represented as the means+SD from three independent experiments. p<0.001.

FIG. 8 demonstrates results of hematoxylin and eosin (H and E) stained skin sections observed under fluorescent microscope at 10× magnification for Naïve control group (a, b, c); IS103 DMSO solution formulation applied group (d, e, f) and vehicle control group (g.h.i) of mice models.

FIG. 9 demonstrates depigmentation effect induced by monobenzone model in mice. During 65 days C57BL/6 mice received monobenzone cream (40%) on specific site of its back and different treatments were giving topically, once a day. On 65^{th} day, depigmentation was evaluated on the monobenzone-exposed site (FIG. 9A) (red circle) and non-exposed site (FIGS. 9B, 9C and 9D) (red arrow and square).

FIGS. 10A to 10D demonstrates levels of cytokines (IL-1β, IL-6, TNF-α, IL-10) in serum samples from monobenzone model were performed with commercial ELISA kit. Each bar represents mean±SEM for 8 animals.

### BRIEF DESCRIPTION OF THE INVENTION

The present invention provides a decapeptide IS103 as a suitable candidate for treatment, prevention and/or amelioration of one or more symptoms of pigmentary disorders. The present invention provides IS103, a synthetic peptide as single therapeutic agent which is an active fragment of bFGF. The peptide significantly induces expression of pigmentation-related genes, such as tyrosinase and increases melanin content in dose-dependent manner.

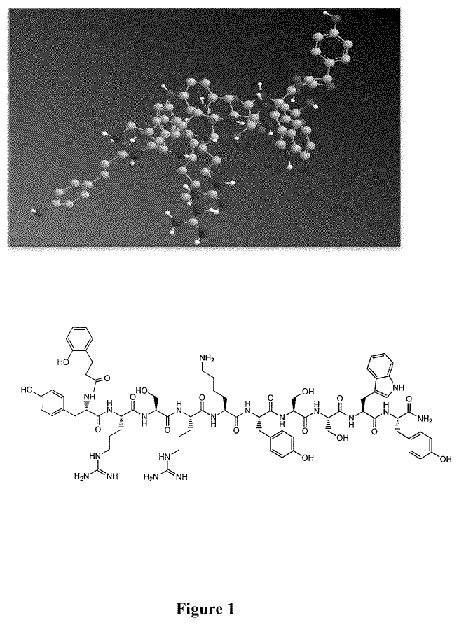

### Synthetic Peptide Is103 (FIG. 1)

Chemical Composition: 10 amino acids

Chemical Formula: C₇₄H₉₈N₁₈O₁₉
Molecular Weight: 1543.679 g/mol
Sequence of the decapeptide

Molecular three letter code:
(SEQ. ID NO 1)
PHPP-Tyr-Arg-Ser-Arg-Lys-Tyr-Ser-Ser-Trp-Tyr-NH₂
Molecular single letter code:
(SEQ. ID No 1)
H-Y-R-S-R-K-Y-S-S-W-Y- NH₂

Molecular primary structure: linear peptide
Molecular secondary structure: β-sheet
Solubility: soluble in water

The formulation of present invention can include variant of IS103 peptide. The variant is a functionally active variant and may be obtained by changing sequence of IS 103 and is characterized by having a biological activity similar to that displayed by IS103 of SEQ. ID NO.1 from which the variant is derived. The variant includes ability of IS103 for treatment, prevention and/or amelioration of one or more symptoms of pigmentary disorders. The functionally active variant of IS103 protein may be obtained by sequence alterations in sequence of IS103, wherein the peptide with the sequence alterations retains function of the unaltered peptide. Such sequence alterations can include, but are not limited to, (conservative) substitutions, deletions, mutations and insertions. The variant can comprise at least 80% of the sequence of IS103, preferably at least 85%, still more preferably at least 90%, even more preferably at least 95% and most preferably at least 97%, 98% or 99%. The variant is derived from the IS103 by at least one amino acid substitution and/or deletion, wherein the functionally active variant has a sequence identity to IS103 of at least 80%, more preferably at least 85%, still more preferably at least 90%, even more preferably at least 95% and most preferably at least 97%, 98% or 99%. The variant of IS103 is functionally active in the context of the present invention, if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of IS103 without sequence alteration. The activity of the variant may be determined or measured as described in the examples and then compared to that obtained for IS103 of the present invention.

The various embodiments of present invention will now herein below be described by means of examples. The following examples are provided solely to illustrate the present invention and are not intended to limit the scope of the invention, as described herein.

### Example 1: Preparation of Decapeptide IS103 by Solid-Phase Peptide Synthesis

The decapeptide IS103 was synthesised by Fmoc solid-phase peptide synthesis method. The peptide chain is assembled stepwise, one amino acid at a time, while attached to an insoluble resin support. This allows the reaction by-products to be removed at each step by simple washing. Amino acids are protected at their amino terminus by the Fmoc (9-fluorenylmethoxycarbonyl) group and coupled to the growing chain after activation of the carboxylic acid terminus. The Fmoc group is then removed by piperidine treatment and the process repeated. After the peptide has been assembled it is removed from the resin by treatment with trifluoroacetic acid (TFA). At the same time, protecting groups on amino acid side chains are also removed yielding the crude linear peptide. Isolation of solid crude peptide was performed by centrifugation. Subsequently purification is performed by preparative HPLC, roto evaporation done for collected pure fraction and then lyophilization performed to get pure peptide.

The peptidyl resin (resin with peptide) containing the peptide of SEQ. ID No. 1 (IS103) with resin was prepared by placing the resin in reaction vessel of the synthesizer and swelling (3 to 4 hours) with DMF; washing the resin with DMF (3 times); two time deprotection by adding 20% piperidine in DMF to the resin and stirring and draining; checking of free amino group availability by ninhydrin test; washing, stirring and draining with DMF (3 times), DCM (2 times) and DMF (3 times); weighing Fmoc Tyr (Fluorenylmethyloxycarbonyl tyrosine), HOBT and solubilizing in DMF to obtain a solution; adding DIC (N,N′-Diisopropyl carbodiimide) to the solution just before adding to the resin; mixing and checking for non-availability of free amino group by ninhydrin test. Formation of clear beads showed that the reaction is complete. The process was repeated with next amino acid sequence to obtain peptidyl resin.

In the synthesis step, coupling cycle is performed for 11 times on manual synthesizer for about 3-4 hours wherein raw materials Fmoc-Lys (Boc)-OH, Fmoc-Tyr (tBu)—OH, Fmoc-Trp (Boc)-OH, Fmoc-Ser (tBu)—OH, Fmoc-Arg (pbf)-OH, 3-Phenylpyrazolo[3,4-d] pyrimidine (PHPP), 1-hydroxy-benzotriazole (HOBT) and carbodiimide (DIC) were mixed. PHPP used for 11th coupling in place of amino acid. After completion of PHPP coupling cycle, peptidyl resins washing, stirring and draining done with DMF (3 times), DCM (2 times), and methanol (3 times). The peptidyl resin was transferred to G2 sintered funnel and washed with diethyl ether. Thereafter the funnel was kept under vacuum for about 7 hours.

Fmoc-deprotection was performed for about 15 minutes and about 50 minutes by using 20% piperidine in DMF. The deprotection completion was verified by ninhydrin test.

In the cleavage step, cleavage cocktail is prepared in round bottom flask, comprising Trifluoroacetic acid (TFA), ethanedithiol (EDT), water, thioanisole, phenol and TIPS (Triisopropylsilane), stirring on a magnetic stirrer for about 20 minutes. Then well mixed cleavage cocktail has been added to another round bottom flask that contains peptidyl resin on a magnetic stirrer. The cleavage was performed for about 5 hours at room temperature. The solution was filtered through G2 sintered funnel and the resin was washed with trifluoroacetic acid (TFA). The filtrates were collected and evaporated in roto evaporator at 40° C. to remove TFA and to reduce the volume to about ⅓ of its original volume. The reduced volume of the filtrate was transferred to round bottom flask and precipitated with chilled diethyl ether. The round bottom flask was swirled for about 30 minutes on a magnetic stirrer and left for settling down the precipitate in the bottom of flask. Once the precipitate was settled the supernatant was decanted and this process was repeated for 4 times more.

The remaining crude peptide was centrifuged in centrifugation tubes, washed with diethyl ether; mix well with glass rod and centrifuge and empty supernatant. This step was performed for 4 times. The crude peptide containing tubes are left for air drying overnight. The powdery compound from the tubes was un-loaded to self-sealed covers, which were placed in PVC container and stored at 2-8° C. Analytical analysis for crude peptide performed as per specification like purity and mass by LC/MS.

Purification was performed by preparative HPLC comprising loading of crude peptide by dissolving it in 20% acetonitrile solution, collecting the fractions of the main peak of the peptide from the fraction collector, checking of purity of fractions by analytical HPLC and polling all the fractions of the pure compounds which are greater than 98% purity; roto evaporating the collected fractions using roto evaporator to remove ACN (acetonitrile). Pooled all the collected fractions whose purity below 98% and above 50%; repeat the above purification process for one time. Thereafter lyophilisation was performed wherein roto evaporated solution was loaded into the glass bottles and lyophilized. The pure peptide was unloaded, weighed, and stored in polypropylene wide mouth bottle at −20° C. for further use. Analytical analysis for pure peptide was performed as per specification like assay and related substances by HPLC, mass by LC/MS etc.

### Example 2: Studying Binding Affinity of IS103 Peptide with MC1R

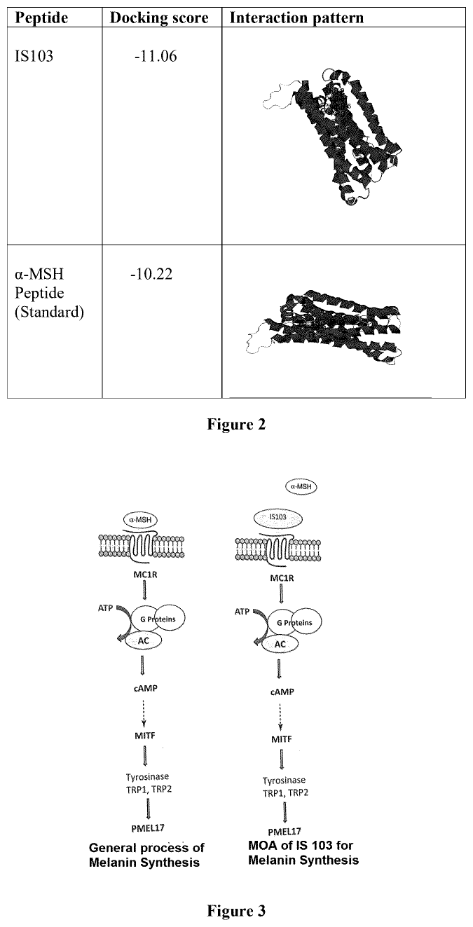

For studying binding affinity of IS103 with MC1R, the sequence IS103 was subjected to in silico docking studies and the proposed mode of action is hypothesized in FIG. 3. α-MSH which binds to MC1R and activates melanin signalling pathway, leads to melanin synthesis and dark brown/black pigmentation. The in silico docking scores of IS103 and α-MSH (as standard) and interaction pattern are shown in FIG. 2.

### CONCLUSION

The in silico study results indicate that IS103 binds with MC1R, blocking the binding of α-MSH to MC1R, thereby inhibiting the melanin synthesis. The study indicates that IS103 is first inhibitor/antagonist for MC1R in melanin signalling.

### Example 3: Preparation of 0.1% W/V IS103 Formulation Solution with DMSO

TABLE 1
WEIGHT PERCENT OF VARIOUS INGREDIENTS
IN THE IS103 DMSO SOLUTION

| S. No. | Ingredients | % w/v |
|---|---|---|
| 1 | Peptide IS103 | 0.05-0.25 |
| 2 | Isopropyl alcohol (IPA) | 10.00-75.00 |
| 3 | Propylene glycol (PEG 400) | 0.10-0.85 |
| 4 | Sucrose | 0.50-4.50 |
| 5 | DMSO | 0.30-5.00 |
| 6 | Water | Quantity sufficient |

### Preparation of Formulation:

1. In a 200 ml glass beaker, required purified water was added and placed on magnetic stirrer set at about 150 rpm. Required quantity of decapeptide (IS103) was added in the beaker and stirred until clear solution was observed.
2. Required quantity of sucrose was added in another glass beaker and stirred until it is completely dissolved. The sucrose solution was added to the clear solution obtained in step 1.
3. In a 500 ml beaker, required quantity of IPA was added and added to clear solution obtained in step 01 and stirred continuously. Further, required quantities of DMSO and propylene glycol were added and stirred for about an hour until clear homogenous solution of the pharmaceutical formulation was formed. The prepared homogenous solution was packed into vials and submitted to quality control for analysis and for further stability studies.

### Example 4: In Vivo Skin Irritation Studies of IS103 Dmso Solution

The level of skin irritation of IS103 loaded DMSO solution formulation was studied on animal models. The animal models used for the study were 08 female BALB/c mice of 6 to 8 weeks obtained from Jeeva Lifesciences and housed at BITS-Pilani, Hyderabad. The mice used for the study were having body weight of 20-30 gm. Three mice were used as naïve control, three mice were treated with blank solution and remaining three mice were treated with IS103 loaded DMSO solution. The study was conducted as per IAEC (Institutional Animal Ethics Committee) protocol number BITS-Hyd/IAEC/2018/12. Skin tolerance tests were done using the Organization for Economic Cooperation and Development guidelines with slit modification. Twenty-four hours before the experiment, fur from backs of all mice were shaved and cleaned, and about 0.5 μm of IS103 loaded DMSO solution was applied on the surface of mice skin for 5 days with once-a-day dosage regimen on 1×1-inch square on dorsum site. The test solution was evenly and gently applied in a test site while untreated skin areas serve as control. The test sites were then examined critically at about 1 hour after the test solution application and at about 24 hours, 48 hours, 72 hours, 7^{th} and 15^{th} day for dermal reaction using Draize scoring criteria. The animals were euthanized on sixth day after daily routine examination and skin was collected and subjected to histopathology. The results of experiment conducted on animal models are produced in Table 2 below.

### RESULTS

### Skin Irritation Assessment

Table 2 showed skin tolerance test findings. Mice showed no irritation signs or skin edema after treatment with 1% solution of IS103-DMSO. The treated skin was intact; no inflammation and erythema compared to untreated site. Edema and erythema score was “0” in each mouse at any time of the observation. This demonstrated that the skin primary irritation index score was 0.

TABLE 2
ERYTHEMA, ESCHAR, EDEMA FORMATION ON MICE SKIN
Mean score of erythema and eschar formation on mice skin

| | Mean Score of erythema and eschar (0 to 4 scale) | | | | | |
|---|---|---|---|---|---|---|
| Group | Day 1 | Day 2 | Day 3 | Day 4 | Day 5 | Day 6 |
| Naive Control | 0 | 0 | 0 | 0 | 0 | 0 |
| Vehicle Control | 0 | 0 | 0 | 0 | 0 | 0 |
| IS103 loaded Solution | 0 | 0 | 0 | 0 | 0 | 0 |

Mean score of edema formation on mice skin

| | Mean Score of edemas (0 to 4 scale) | | | | | |
|---|---|---|---|---|---|---|
| Group | Day 1 | Day 2 | Day 3 | Day 4 | Day 5 | Day 6 |
| Naive Control | 0 | 0 | 0 | 0 | 0 | 0 |
| Vehicle Control | 0 | 0 | 0 | 0 | 0 | 0 |
| IS103 loaded Solution | 0 | 0 | 0 | 0 | 0 | 0 |

### CONCLUSIONS

The results indicate safety profile of the IS103 loaded DMSO solution of present invention when applied topically.

### Histological Studies for IS103 Formulation

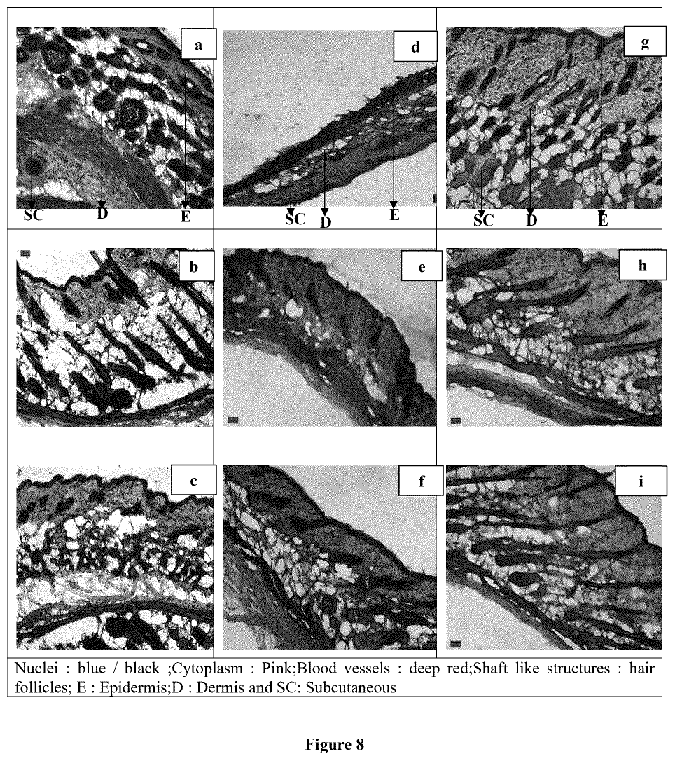

Mice models for skin irritation studies shed important light into pathogenesis of skin inflammation. The inventor of present invention highlighted cryosections of skin surface stained with hematoxylin and eosin staining (H and E staining) of the mice models of all groups including IS103 loaded vehicle controls as well. The pictures (FIG. 8) show that all the H and E stained transverse cryo-sections for all the animal groups were consistent when compared to that of the naïve control group (FIG. 8).

### CONCLUSIONS

The results of histological study show no mechanical injury or skin disruption on application of IS103 loaded DMSO solution and placebo formulation. The results further substantiate non-irritant and safety profile of IS103 loaded DMSO solution formulation applied on Balb/c mouse models. The results of skin irritation study and histological study of the formulation demonstrate absence of any significant cytotoxicity along with no significant effect on cell viability.

The findings of present invention indicate that formulation of IS103 with 0.1% solution showed good physicochemical properties and stability, thus providing a safe and stable solution delivery system. The prepared formulation solution was safe to use on skin. The present invention provides method of preparation and characterization of topical IS103 peptide with DMSO solution formulation. The IS103 DMSO solution of present invention can be prepared successfully for industrial application as it simultaneously satisfies stability and safety criteria as solution and is an excellent alternative to the currently available formulations for vitiligo.

### Example 5: Stability of IS103 Api (Active Pharmaceutical Ingredient or Drug Substance) and IS103 DMSO 0.1% Solution (Drug Product) Under Accelerated Conditions and Under Long-Term Storage Conditions

### 5.1: Stability of Is103 Api

### INTRODUCTION

The purpose of stability testing is to provide evidence on how the quality of a drug substance or drug product varies with time under the influence of a variety of environmental factors such as temperature, humidity, etc. and to establish a re-test period for the drug substance or a shelf life for the drug product and recommended storage conditions. The study was conducted according to the WHO and schedule-Y guidelines [20-21].

### Container Closure System

The IS103 API is a lyophilized white fluffy powder, and it was filled at sterile condition in glass injection vial which has airlock-rubber capping. This container closure system is same as or simulates the packaging proposed for storage and distribution.

### Specifications

The study specifications were designed and conducted according to WHO and Schedule Y guidelines [20-21] and the details of the same were described in below section.

### Test Attributes and Acceptance Criteria

This study has included testing of those attributes of the IS103 API that are susceptible to change during storage and are likely to influence quality, safety and/or efficacy. The testing was covered, as appropriate, the physical and chemical attributes.

In this stability testing of IS103 API, the below attributes were investigated
which are potentially susceptible to change during the course of storage
which are likely to influence quality, safety and/or efficacy.

The following test attributes have been selected according to the above said guidelines:
appearance; odour; colour; colour of solution (when IS103 API is soluble in solvent); clarity of solution; degradation and assessment of container closure system.

TABLE 3
ATTRIBUTES TESTED FOR IS103 API
AND THEIR ACCEPTANCE CRITERIA

| S. No. | Test attributes | Acceptance criteria |
|---|---|---|
| 1 | Appearance | White to off-white substance |
| 2 | Odour | Odour less |
| 3 | Colour | White |
| 4 | Colour of the solution | Clear Solution |
| 5 | Clarity of the solution | Clear Transparent |
| 6 | Degradation | Not more than 5% |
| 7 | Assessment of container closure | Appearance unchanged |
| | system | |

### Testing Frequency

Testing frequency for long term studies have been designed as per schedule Y. The long-term stability studies have been conducted on 1, 3, 6, 9, 12, 18, 24 and 36 months.

### Storage Conditions

The studies were conducted under appropriate storage conditions (with appropriate tolerances) that test IS103 API thermal stability and its sensitivity to moisture. The IS103 API is intended for storage for 36 months. Accordingly, long term stability studies were done for 36 months. The details of the storage conditions have been furnished hereunder.

TABLE 4
SAMPLE STORAGE CONDITIONS AND FREQUENCY
OF TESTING AS PER SCHEDULE Y REQUIREMENT

| | | Storage conditions | |
|---|---|---|---|
| | | Condition 1 | |
| | | (Accelerated | Condition 2 |
| S. No. | Parameter | studies) | (Long term studies) |
| 1. | Temperature | 5 ± 3° C. | −20 ± 5° C. |
| 2. | Relative Humidity | 60 ± 5% | 60 ± 5% |
| 3. | Frequency of testing | 1, 2, 3, 4, 5 and 6 | 1, 3, 6, 9, 12, 18, 24 |
| | (months) | | and 36 |

### Stability Commitment

The stability data was generated on pilot scale batches. It has been proposed that the drug substance re-test period for 36 months.

### Evaluation of Results

### 5.1.1: Acceleratory Stability Evaluation (Condition 1)

The attributes were not changed during the acceleratory stability testing period. The details of the same were furnished below. The attributes tested for IS103 API are appearance, color, odor, color of solution, clarity of solution and assessment of container closure system. IS103 API was observed in its natural color and no change was observed in all other attributes during the study period. All other attributes were noted within acceptance range during the study period.

All the above mentioned attributes were observed within the acceptance range as shown in Table 5.

TABLE 5
EVALUATION OF INTERNAL CHANGE IN ATTRIBUTES DURING
ACCELERATORY (CONDITION 1) STUDY PERIOD

| Attributes | 1^{st} month | 2^{nd} month | 3^{rd} month | 6^{th} month |
|---|---|---|---|---|
| Appearance | No change | No change | No change | No change |
| Color | No change | No change | No change | No change |
| Odor | No change | No change | No change | No change |
| Color of solution | No change | No change | No change | No change |
| Clarity of solution | No change | No change | No change | No change |
| Assessment of container | No change | No change | No change | No change |
| closure system | | | | |

### 5.1.2: Long Term Stability Evaluation (Condition 2)

The attributes described in above section were not changed during the long term stability study period. The details of the same have been furnished below table. Attributes of drug substance tested were appearance, color, odor, color of solution, clarity of solution and assessment of container closure system.

All the above mentioned attributes were observed within the acceptance range as shown in Table 6.

TABLE 6
EVALUATION OF INTERNAL CHANGE IN ATTRIBUTES
DURING THE LONG TERM STUDY PERIOD

| Attributes | 1^{st} month | 3^{rd} month | 12^{th} month | 18^{th} month | 24^{th} month | 36^{th} month |
|---|---|---|---|---|---|---|
| Appearance | No change | No change | No change | No change | No change | No change |
| Color | No change | No change | No change | No change | No change | No change |
| Odor | No change | No change | No change | No change | No change | No change |
| Color of solution | No change | No change | No change | No change | No change | No change |
| Clarity of solution | No change | No change | No change | No change | No change | No change |
| Assessment of | No change | No change | No change | No change | No change | No change |
| container closure | | | | | | |
| system | | | | | | |

### 5.1.3: Accelerated Stability Study Results

Name of the product: IS103

API Batch size: 200 m·mol
Storage conditions: 5±3° C.

TABLE 7
STORAGE STABILITY OF IS103 API AT ACCELERATED STABILITY CONDITIONS (CONDITION 1)

| | | | Identification | | | | | |
|---|---|---|---|---|---|---|---|---|
| | | | (By HPLC) | | | | | |
| | | | Retention time | | | API | | |
| | | | of main peak | | | calculated | | |
| | | | in the | | | on the | | |
| | | | chromatogram | | | anhydrous | | |
| | | | of the sample | | Water | basis | | |
| | | | preparation | Identification | Content | Not less | | |
| | | | should | Mass | (By Karl | than | Related substances | |
| | Appearance | | correspond to | Spectrum | Fischer- | 98.0% and | (By HPLC) | |
| | white to off | Solubility | that of standard | (By LC/MS) | Titrator) | not more | Any Max | Total |
| Interval | white | Sparingly | preparation as | Should be | Not be | than | Impurity | Impurity |
| in | amorphous | soluble in | obtained in the | 1542.7 ± 2 | more than | 102.0% | NMT | NMT |
| Months | powder | water | assay. | M.W. | 5.0% w/w | (OAB) | 2.0% | 4.0% |
| Initial | White | Complies | Complies | 1543.3 M.W. | 4.18% w/w | 99.73% | 0.39% | 2.09% |
| | amorphous | | | | | | | |
| | powder | | | | | | | |
| 1 M | White | Complies | Complies | 1543.4 M.W. | 4.27% w/w | 99.68% | 0.35% | 2.23% |
| | amorphous | | | | | | | |
| | powder | | | | | | | |
| 3 M | White | Complies | Complies | 1543.4 M.W. | 4.29% w/w | 99.14% | 0.40% | 2.30% |
| | amorphous | | | | | | | |
| | powder | | | | | | | |
| 6 M | White | Complies | Complies | 1543.5 M.W. | 4.87% w/w | 99.00% | 0.49% | 2.44% |
| | amorphous | | | | | | | |
| | powder | | | | | | | |

* NMT is “not more than” and NLT is “not less than”

Conclusion: IS103 API is found to be stable for 6 months at accelerated condition as no significant change from initial analysis has been observed.

### 5.1.4: Long Term Stability Study Results

Name of the product: IS103 API

Batch size: 200 m·mol
Storage conditions: −20±5° C.

TABLE 8
STORAGE STABILITY OF IS103 API AT LONG-TERM CONDITIONS (CONDITION 2)

| | | | Identification | | | | | |
|---|---|---|---|---|---|---|---|---|
| | | | (By HPLC) | | | | | |
| | | | Retention time | | | | | |
| | | | of main peak | | | API | | |
| | | | in the | | | calculated | | |
| | | | chromatogram | | | on the | | |
| | | | of the sample | | Water | anhydrous | | |
| | | | preparation | Identification | Content | basis | | |
| | | | should | Mass | (By Karl | Not less | Related substances | |
| | Appearance | | correspond to | Spectrum | Fischer- | than | (By HPLC) | |
| | white to off | Solubility | that of standard | (By LC/MS) | Titrator) | 98.0% and | Any Max | Total |
| Interval | white | Sparingly | preparation as | Should be | Not be | not more | Impurity | Impurity |
| in | amorphous | soluble in | obtained in the | 1542.7 ± 2 | more than | than | NMT | NMT |
| Months | powder | water | assay. | M.W. | 5.0% w/w | 102.0% | 2.0% | 4.0% |
| Initial | White | Complies | Complies | 1541.7 M.W. | 3.91% w/w | 99.73% | 0.14% | 0.32% |
| | amorphous | | | | | | | |
| | powder | | | | | | | |
| 3 M | White | Complies | Complies | 1542.2 M.W. | 4.06% w/w | 99.51% | 0.22% | 0.37% |
| | amorphous | | | | | | | |
| | powder | | | | | | | |
| 12 M | White | Complies | Complies | 1542.3 M.W. | 4.45% w/w | 99.11% | 0.36% | 0.44% |
| | amorphous | | | | | | | |
| | powder | | | | | | | |
| 18 M | White | Complies | Complies | 1542.3 M.W. | 4.54% w/w | 99.03% | 0.62% | 0.62% |
| | amorphous | | | | | | | |
| | powder | | | | | | | |
| 24 M | White | Complies | Complies | 1543.4 M.W. | 4.61% w/w | 98.95% | 0.60% | 0.82% |
| | amorphous | | | | | | | |
| | powder | | | | | | | |
| 36 M | White | Complies | Complies | 1542.6 M.W. | 4.56% w/w | 98.73% | 0.84% | 0.97% |
| | amorphous | | | | | | | |
| | powder | | | | | | | |

* NMT is “not more than” and NLT is “not less than”

Conclusion: For drug substances or products intended for storage in a freezer, the retest period or shelf life should be based on long-term data [21]. IS103 API is found to be stable for 36 months at long term stability condition (of −20±5° C.) as no significant change from initial analysis has been observed.

The ICH guidelines [22, 23] and schedule Y provides that for a drug substances intended for storage in a refrigerator or freezer, the drug should pass long term stability storage conditions and accelerated storage study conditions for minimum time period [21]. Based on all the above data illustrated in Tables 13 to 16 it can be concluded that the drug substance is stable at refrigerator (5±3° C.) conditions for 36 months.

### 5.2: Stability of Drug Product-IS103 Dmso-0.1% W/V Solution

### INTRODUCTION

The purpose of stability testing is to provide evidence on how the quality of a drug product varies with time under the influence of a variety of environmental factors such as temperature, humidity, etc and to establish a shelf life for the drug product and recommended storage conditions. The study was conducted according to the WHO and Schedule-Y guidelines [20-21].

### General Properties

The drug product is IS103 DMSO −0.1% w/v solution formulation containing IS103, active pharmaceutical ingredient (API). The drug substance, IS103, is a white amorphous lyophilized powder and is an applicant's designation for a 10 amino acid peptide developed and formulated for treatment of Vitiligo.

### Example 5.2.1: Composition of Is103 DMSO −0.1% W/V Solution

The list of components, with its quantities, usages in process and their quality standers were furnished hereunder.

TABLE 9
WEIGHT PERCENT OF VARIOUS INGREDIENTS
IN THE IS103 DMSO SOLUTION

| S. No. | Ingredients | % w/v |
|---|---|---|
| 1 | Peptide IS103 | 0.05-0.25 |
| 2 | Isopropyl Alcohol (IPA) | 10.00-75.00 |
| 3 | Propylene Glycol (PEG 400) | 0.10-0.85 |
| 4 | Sucrose | 0.50-4.50 |
| 5 | DMSO | 0.30-5.00 |
| 6 | Water | Quantity sufficient |

### Selection of Batches

The IS103 DMSO solution was manufactured by ISSAR pharma. Three batches were manufactured at pilot scale level by the same synthetic route as and using a method of manufacture and procedure that simulates the final process to be used for, production batches. The overall quality of the batches of drug product placed on these stability studies will be representative of the quality of the material to be made on a production scale.

### Container Closure System

Amber color vial will be used as primary packing material. The vial should be fitted with a screw cap closure that minimizes microbial contamination.

### Specifications

The study specifications were designed and conducted according to WHO and Schedule Y guidelines [20-21] and the details of the same were described in below section.

### Test Attributes and Acceptance Criteria

This study has included testing of those attributes of the drug product that are susceptible to change during storage and are likely to influence quality, safety and/or efficacy. The testing was covered, as appropriate, the physical and chemical attributes.

In this stability testing of drug product, the below attributes were investigated
which are potentially susceptible to change during the course of storage
which are likely to influence quality, safety and/or efficacy.

The following test attributes have been selected according to the above said guidelines.
appearance; odour; colour; degradation and assessment of container closure system.

TABLE 10
ATTRIBUTES TESTED FOR IS103 DMSO SOLUTION
AND THEIR ACCEPTANCE CRITERIA

| S. No. | Test attributes | Acceptance criteria |
|---|---|---|
| 1 | Appearance | Colorless/Transparent solution |
| 2 | Odour | Odour less |
| 3 | Colour | Transparent |
| 4 | Degradation | Not more than 5% |
| 5 | Assessment of container closure | Appearance unchanged |
| | system | |

### Testing Frequency

Testing frequency for long term studies have been designed as per Schedule Y. The long term stability studies have been conducted on 1, 3, 6, 9, 12, 18, 24 and 36 months.

### Storage Conditions

The studies were conducted under appropriate storage conditions (with appropriate tolerances) that test drug product thermal stability and its sensitivity to moisture. The drug product is intended to be stored for 24 months. Accordingly, long term stability studies were done for 36 months, as per the guidelines the Applicant has to present data additionally for one more year. The details of the storage conditions have been furnished hereunder.

TABLE 11
SAMPLE STORAGE CONDITIONS AND FREQUENCY OF TESTING
AS PER SCHEDULE Y REQUIREMENT [20-21]

| | | Storage Conditions | | |
|---|---|---|---|---|
| | | Condition 1 | Condition 2 | Condition 3 |
| S. No. | Parameter | (Accelerated Studies) | (Long term Studies) | (Long term studies) |
| 1. | Temperature | 40° C. ± 2° C. | 30° C. ± 2° C. | 25° C. ± 2° C. |
| 2. | Relative | 75% RH ± 5% | 65% RH ± 5% | 65% RH ± 5% |
| | humidity | | | |
| 3. | Frequency of | 1, 2, 3, 4, 5, and 6 | 1, 3, 6, 9, 12, 18, 24, and 36 | 1, 3, 6, 9, 12, 18, 24, |
| | Testing (months) | | | and 36 |

### Evaluation of Results

### 5.2.2: Acceleratory Stability Evaluation (Condition 1)

The attributes were not changed during the acceleratory stability testing period. The details of the same were furnished below.

Appearance, color, odor, color of solution, clarity of solution and assessment of container closure system.

Drug product was observed in its natural color and no change was observed in all other attributes during the study period. All other attributes were noted within acceptance range during the study period.

TABLE 12
EVALUATION OF INTERNAL CHANGE IN ATTRIBUTES DURING
THE ACCELERATORY (CONDITION 1) STUDY PERIOD

| Attributes | 1^{st} month | 2^{nd} month | 3^{rd} month | 6^{th} month |
|---|---|---|---|---|
| Appearance | No change | No change | No change | No change |
| Color | No change | No change | No change | No change |
| Odor | No change | No change | No change | No change |
| Assessment of container | No change | No change | No change | No change |
| closure system | | | | |

### 5.2.3: Long Term Stability Evaluation (Condition 2)

The attributes described in above section were not changed during the long term stability study period. The details of the same have been furnished below table.

Appearance, color, odor, color of solution, clarity of solution and assessment of container closure system.

All mentioned attributes in below table were observed within the acceptance range.

TABLE 13
EVALUATION OF INTERNAL CHANGE IN ATTRIBUTES DURING
THE LONG TERM STUDY (CONDITION 2) PERIOD

| | 1^{st} | 3^{rd} | 6^{th} | 9^{th} | 12^{th} | 18^{th} | 24^{th} | 36^{th} |
|---|---|---|---|---|---|---|---|---|
| Attributes | month | month | month | month | month | month | month | month |
| Appearance | No | No | No | No | No | No | No | No |
| | change | change | change | change | change | change | change | change |
| Color | No | No | No | No | No | No | No | No |
| | change | change | change | change | change | change | change | change |
| Odor | No | No | No | No | No | No | No | No |
| | change | change | change | change | change | change | change | change |
| Assessment of | No | No | No | No | No | No | No | No |
| container | change | change | change | change | change | change | change | change |
| closure system | | | | | | | | |

### 5.2.4: Long Term Stability Evaluation (Condition 3)

The attributes described in above section were not changed during the long term stability study period. The details of the same have been furnished below table.

Appearance, color, odor, color of solution, clarity of solution and assessment of container closure system.

All mentioned attributes in below table were observed within the acceptance range.

TABLE 14
EVALUATION OF INTERNAL CHANGE IN ATTRIBUTES DURING
THE LONG TERM STUDY (CONDITION 3) PERIOD

| | 1^{st} | 3^{rd} | 6^{th} | 9^{th} | 12^{th} | 18^{th} | 24^{th} | 36^{th} |
|---|---|---|---|---|---|---|---|---|
| Attributes | month | month | month | month | month | month | month | month |
| Appearance | No | No | No | No | No | No | No | No |
| | change | change | change | change | change | change | change | change |
| Color | No | No | No | No | No | No | No | No |
| | change | change | change | change | change | change | change | change |
| Odor | No | No | No | No | No | No | No | No |
| | change | change | change | change | change | change | change | change |
| Assessment of | No | No | No | No | No | No | No | No |
| container | change | change | change | change | change | change | change | change |
| closure system | | | | | | | | |

### 5.2.5: Accelerated Stability Study Results

Name of the product: IS103 DMSO −0.1% w/v solution

Batch size: 500 ml
Pack size: 5 ml
Storage conditions: 40±2° C.

TABLE 15
STORAGE STABILITY OF IS103 DMSO SOLUTION AT ACCELERATORY STABILITY CONDITIONS (CONDITION 1)

| | | Stability Tests and Limits | | | | | | |
|---|---|---|---|---|---|---|---|---|
| | | | Identification | | | | | Microbiological Limits |
| | | | (by HPLC) | | | | | Total aerobic Count: |
| | | | The retention time | | | | | NMT 100 cfu/ml Yeast |
| | | | of the major peak in | | | | | and Molds: 10 cfu/ml |
| | | | the chromatogram | | Assay | | | Absence of E. Coli, |
| | | Appearance | of the test predation | pH | (By HPLC) | Related Substance | | S. aureus, P. aeruginosa, |
| | | Clear | should correspond | (at 25° C.) | NLT 90.0% | (By HPLC) | | Salmonella, Bile-tolerant |
| | | solution with a | to the standard | Between | and NMT | Single | | gram negative bacteria, |
| | Stability | characteristic | preparation, as | 3.0 and | 110.0% of | Maximum | Total | clostridia and candida |
| S. No | station | odor | obtained in the assay | 6.0 | label claim | Impurity | Impurities | albicans |
| 1 | Initial | Complies | Sample RT complies | 4.02 | 111.23% | 0.021% | 0.050% | Complies |
| | | | with Standard RT | | | | | |
| 2 | 1 M | Complies | Sample RT complies | 4.65 | 108.40% | 0.007% | 0.090% | Complies |
| | | | with Standard RT | | | | | |
| 3 | 2 M | Complies | Sample RT complies | 4.92 | 104.30% | 0.017% | 0.099% | Complies |
| | | | with Standard RT | | | | | |
| 4 | 3 M | Complies | Sample RT complies | 4.90 | 101.89% | 0.026% | 1.106% | Complies |
| | | | with Standard RT | | | | | |
| 5 | 6 M | Complies | Sample RT complies | 5.34 | 96.95% | 0.142% | 1.958% | Complies |
| | | | with Standard RT | | | | | |

* NMT is “not more than” and NLT is “not less than”

Conclusions: IS103 DMSO −0.1% w/v solution is found to be stable for 6 months at accelerated condition as no significant change from initial analysis has been observed.

### 5.2.6: Long Term Stability Study Results Condition-02

Name of the product: IS103 DMSO −0.1% w/v solution

Batch size: 500 ml Pack size: 5 ml
Storage conditions: 30±2° C.

TABLE 16
STORAGE STABILITY OF IS103 DMSO SOLUTION AT LONG TERM STABILITY CONDITIONS (CONDITION 2)

| | | Stability Tests and Limits | | | | | | |
|---|---|---|---|---|---|---|---|---|
| | | | Identification | | | | | Microbiological Limits |
| | | | (by HPLC) | | | | | Total aerobic Count: |
| | | | The retention time | | | | | NMT 100 cfu/ml Yeast |
| | | | of the major peak in | | | | | and Molds: 10 cfu/ml |
| | | | the chromatogram | | Assay | | | Absence of E. Coli, |
| | | Appearance | of the test predation | pH | (By HPLC) | Related Substance | | S. aureus, P. aeruginosa, |
| | | Clear | should correspond | (at 25° C.) | NLT 90.0% | (By HPLC) | | Salmonella, Bile-tolerant |
| | | solution with a | to the standard | Between | and NMT | Single | | gram negative bacteria, |
| | Stability | characteristic | preparation, as | 3.0 and | 110.0% of | Maximum | Total | clostridia and candida |
| S. No | station | odor | obtained in the assay | 6.0 | label claim | Impurity | Impurities | albicans |
| 1 | Initial | Complies | Sample RT complies | 4.02 | 111.23% | 0.021% | 0.050% | Complies |
| | | | with Standard RT | | | | | |
| 2 | 3 M | Complies | Sample RT complies | 4.02 | 101.81% | 0.021% | 0.264% | Complies |
| | | | with Standard RT | | | | | |
| 3 | 6 M | Complies | Sample RT complies | 5.10 | 99.94% | 0.206% | 1.189% | Complies |
| | | | with Standard RT | | | | | |
| 4 | 9 M | Complies | Sample RT complies | 4.98 | 99.67% | 0.326% | 1.106% | Complies |
| | | | with Standard RT | | | | | |
| 5 | 12 M | Complies | Sample RT complies | 4.68 | 99.06% | 0.424% | 1.158% | Complies |
| | | | with Standard RT | | | | | |
| 6 | 18 M | Complies | Sample RT complies | 4.25 | 103.12% | 0.521% | 1.264% | Complies |
| | | | with Standard RT | | | | | |
| 7 | 24 M | Complies | Sample RT complies | 4.32 | 99.34% | 0.427% | 1.077% | Complies |
| | | | with Standard RT | | | | | |
| 8 | 36 M | Complies | Sample RT complies | 5.35 | 100.76% | 0.520% | 1.517% | Complies |
| | | | with Standard RT | | | | | |

* NMT is “not more than” and NLT is “not less than”

Conclusion: IS103 DMSO −0.1% w/v solution is found to be stable for 36 months at long term stability condition as no significant change from initial analysis has been observed.

### 5.2.7: Long Term Stability Study Results Condition-03

Name of the product: IS103DMSO −0.1% w/v solution

Batch size: 500 ml
Pack size: 5 ml
Storage conditions: 25±2° C.

TABLE 17
STORAGE STABILITY OF IS103 DMSO SOLUTION AT LONG TERM STABILITY CONDITIONS (CONDITION 3)

| | | Stability Tests and Limits | | | | | | |
|---|---|---|---|---|---|---|---|---|
| | | | Identification | | | | | Microbiological Limits |
| | | | (by HPLC) | | | | | Total aerobic Count: |
| | | | The retention time | | | | | NMT 100 cfu/ml Yeast |
| | | | of the major peak in | | | | | and Molds: 10 cfu/ml |
| | | Appearance | the chromatogram | | Assay | | | Absebce of E. Coli, |
| | | Clear | of the test predation | pH | (By HPLC) | Related Substance | | S. aureus, P. aeruginosa, |
| | | solution | should correspond | (at 25° C.) | NLT 90.0% | (By HPLC) | | Salmonella, Bile-tolerant |
| | | with a | to the standard | Between | and NMT | Single | | gram negative bacteria, |
| | Stability | characteristic | preparation, as | 3.0 and | 110.0% of | Maximum | Total | clostridia and candida |
| S. No | station | odor | obtained in the assay | 6.0 | label claim | Impurity | Impurities | albicans |
| 1 | Initial | Complies | Sample RT complies | 4.02 | 111.23% | 0.021% | 0.050% | Complies |
| | | | with Standard RT | | | | | |
| 2 | 3 M | Complies | Sample RT complies | 4.02 | 101.30% | 0.015% | 0.201% | Complies |
| | | | with Standard RT | | | | | |
| 3 | 6 M | Complies | Sample RT complies | 4.76 | 99.20% | 0.094% | 1.046% | Complies |
| | | | with Standard RT | | | | | |
| 4 | 9 M | Complies | Sample RT complies | 4.70 | 99.94% | 0.226% | 1.106% | Complies |
| | | | with Standard RT | | | | | |
| 5 | 12 M | Complies | Sample RT complies | 5.34 | 96.95% | 0.424% | 1.258% | Complies |
| | | | with Standard RT | | | | | |
| 6 | 18 M | Complies | Sample RT complies | 4.56 | 102.73% | 0.515% | 1.231% | Complies |
| | | | with Standard RT | | | | | |
| 7 | 24 M | Complies | Sample RT complies | 4.32 | 98.23% | 0.549% | 1.375% | Complies |
| | | | with Standard RT | | | | | |
| 8 | 36 M | Complies | Sample RT complies | 5.15 | 100.32% | 0.506% | 1.490% | Complies |
| | | | with Standard RT | | | | | |

* NMT is “not more than” and NLT is “not less than”

Conclusions: IS103 DSMO −0.1% solution is found to be stable for 36 months at long term stability condition as no significant change from initial analysis has been observed.

Statements/Labeling: The drug product is stable for 24 months at room temperature.

### Conclusion of Stability Studies for IS103 Dmso −0.1% W/V Solution

The drug product, IS103 DMSO −0.1% solution is a synthetic peptide based formulation and is available in liquid form. It was intended to store the drug product for 24 months at room temperature conditions. Hence long-term studies were carried out for 36 months at Conditions 1 and 2 as per the guidelines. The batches used for stability studies were manufactured at pilot scale level by the same synthetic route as and using a method of manufacture and procedure that simulates the final process to be used for, production batches.

Attributes like appearance, color, odor and assessment of container closure system were not changed under long term storage condition during 24 months period. However a slight degradation is observed during 24 months under long term storage condition but this change did not cross significant change limit. Rate of degradation and statistical regression line were observed within the acceptance criteria during the long term study period. It indicates that the drug product is stable at room temperature conditions for long duration.

0.1% w/v of IS103 solution formulation with DMSO was tested for stability under accelerated conditions of temperature and humidity at time interval of 3, 6, 9, 12, 18 and 24 months. For indicating stability, the retention time of major peak in chromatogram of the test formulation should corresponded to chromatogram of standard formulation, as obtained in the assay. Assay results at accelerated conditions of 30±2° C. at 65±5% RH; 25±2° C. at 60±5% RH and 40±2° C. at 75±5% RH (Conditions 1 to 3) meet acceptance criteria even after 24-month storage. Results of microbial enumeration test and test for specified microorganisms were also acceptable. The results indicate high shelf life and storage stability of IS103 solution formulation at even accelerated conditions of temperature and humidity. The results indicate that the 0.1% w/v of IS103 DMSO formulation of present invention is better than other formulations.

Thus, the IS103 solution formulation of present invention are suitable candidates for treating pigmentary diseases with low cytotoxicity, reduced side effects on cell viability along with high shelf-life and storage capability.

Based on all the above it can be concluded that the drug substance is stable at room temperature condition for 24 months.

### Example 6: Cell Viability, Melanin Content and In Vitro Tyrosinase Activity of Decapeptide

Cell lines used for study: The inventor of present invention studied in vitro tyrosinase activity and melanin activity of decapeptide IS103 on B16F10 melanoma cell lines procured from ATCC with product code ATCC® CRL-6475™ and NHEM (Normal Human Epidermal Melanocytes) melanoma cell lines procured from PromoCell with product code C-12403.

Culture and maintenance of cell lines: B16F10 cells were cultured in RPMI containing 10% fetal bovine serum, 100 units per millilitre (U/mL) penicillin, 0.1 mg/mL streptomycin, and 0.25 microgram per millilitre (μg/mL) amphotericin B at 37 degrees Celsius in a humidified 95% air/5% CO₂ incubator. Drug treatment was done 24 hours after seeding. Cells were harvested 48 hours later and melanin content and tyrosinase activity determined in triplicate. NHEM cells were cultured in DMEM containing 10% fetal bovine serum, 100 units per milliliter (U/mL) penicillin, 0.1 mg/mL streptomycin, and 0.25 microgram per millilitre (μg/mL) amphotericin B at 37 degrees Celsius in a humidified 95% air/5% CO₂ incubator. Drug treatment was done 24 hours after seeding. Cells were harvested 48 hours later and melanin content and tyrosinase activity determined in triplicate.

IS103 was dissolved in water for injection or DMSO to prepare stock solution of about 50 mg/ml. The stock solution was used for preparation of subsequent dilutions in solvent to achieve working dilutions for treatment ranging 0.1 μg/ml to 1000 μg/ml. Dilutions of IS103 prepared were 0.1, 1, 10, 50, 100, 250, 500 and 1000 μg/ml. chemicals and reagents used for the study were Mushroom tyrosinase, L-3,4-dihydroxyphenylalanine (L-DOPA), 3-isobutyl-1-methylxanthine (IBMX), and α-Melanocyte Stimulating Hormone (α-MSH).

### Example 6.1: Cell Viability and Melanin Content Assay in B16F10 and NHEM Cell Lines

B16F10 and NHEM cells were sub-cultured into 96-well culture plates at a density of 1×10⁴ cells/well in 100 μL medium and after about 24 hours of incubation, the medium was discarded and replaced with 100 μL of a suitable medium (RPMI for B16F10 and DMEM for NHEM with 1% FBS) for starvation and then treated in the presence or absence of various concentrations of test compound and positive controls (IBMX and α-MSH). The plates were incubated in a 37° C. humidified incubator in a 5% CO₂ atmosphere for about 24 hours. At the end of the incubation, about 50 μL of XTT test solution prepared by mixing about 5 mL of XTT-labeling reagent and about 100 μL of electron coupling reagent is added to each well and after about 4 hours of incubation at 37° C. and a about 5% CO₂ incubator, the absorbance was measured on an ELISA reader at a test wavelength of 490 nm.

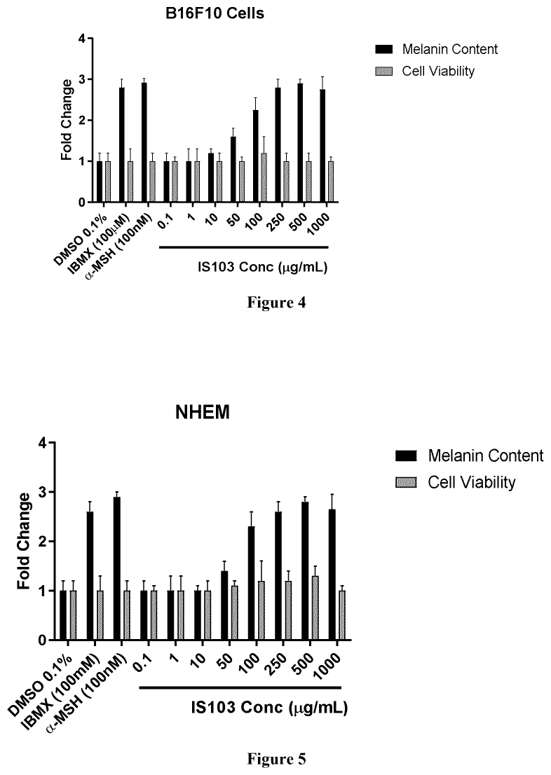

Cells were seeded in a 6-well plate (2×10⁴ cells/well) and incubated overnight to allow the cells to adhere. After treating with various test samples for 4 or 5 days, cells were washed with phosphate-buffered saline (PBS), trypsinized, counted, and then lysed in 1 N NaOH at 100° C. for about 1 hour. The melanin content in each sample was calculated by comparison of absorbance at 400 nm of unknown samples with a standard curve obtained using synthetic melanin. IS103 enhances melanin synthesis in a dose dependent manner without significant toxicity. B16F10 and NHEM cells were treated with different concentrations of IS103 ranging from 0.1 to 1000 μg/ml and analysed melanin contents in cells. It was observed that IS103 was able to enhance pigmentation of B16F10 and NHEM cells in a dose-dependent manner (Table 18 and FIGS. 4 and 5). The concentration of IS103 at 10 μg/ml was sufficient to significantly enhance melanin synthesis. To assess the toxicity of the IS103 peptide, cell viability was analyzed by MTT assay.

The results revealed that treatment of the B16F10 and NHEM cells with 1000 μg/ml of IS103 for about 48 hours did not exert any significant effect on cell viability (FIGS. 4 and 5). These results demonstrate that IS103 enhances melanin synthesis in a dose dependent manner without significantly affecting cell viability.

TABLE 18
MELANIN CONTENT IN B16F10 AND NHEM CELLS

| | B16F10 CELLS | | NHEM CELLS | |
|---|---|---|---|---|
| | | % increase in | | % increase in |
| | | melanin content | | melanin content |
| | Fold change in | compared to | Fold change in | compared to |
| Stimulants | melanin content | control | Melanin Content | control |
| Control (DMS0 0.1%) | 1.0 ± 0.20 | 0 | 1.0 ± 0.20 | 0 |
| IBMX (100 μM) | 2.8 ± 0.20 | 180 | 2.6 ± 0.20 | 160 |
| α- MSH (100 nM) | 2.9 ± 0.10 | 192 | 2.9 ± 0.20 | 190 |
| IS103 (0.1 μg/mL) | 1.0 ± 0.20 | 0 | 1.0 ± 0.20 | 0 |
| IS103 (1 μg/mL) | 1.0 ± 0.30 | 0 | 1.0 ± 0.20 | 0 |
| IS103 (10 μg/mL) | 1.2 ± 0.10 | 20 | 1.0 ± 0.20 | 0 |
| IS103 (50 μg/mL) | 1.6 ± 0.20 | 60 | 1.4 ± 0.20 | 40 |
| IS103 (100 μg/mL) | 2.3 ± 0.30 | 125 | 2.3 ± 0.20 | 130 |
| IS103 (250 μg/mL) | 2.8 ± 0.20 | 180 | 2.6 ± 0.20 | 160 |
| IS103 (500 μg/mL) | 2.9 ± 0.10 | 190 | 2.8 ± 0.20 | 180 |
| IS103 (1000 μg/mL) | 2.8 ± 0.30 | 176 | 2.7 ± 0.20 | 165 |

Results shown in the table 18 are Mean ± SD obtained from triplicate experiments.

### CONCLUSIONS

B16F10 cells: IS103 (0.1 μg/mL-1000 μg/mL) demonstrated 0.0%-176% increase in melanin content when compared to control. IS103 concentration at 250 μg/mL (180%) μg/mL and 500 μg/mL (190%) showed similar results of standard positive controls IBMX (180%) and α-MSH (192%).

NHEM cells: IS103 (0.1 μg/mL-1000 μg/mL) demonstrated 0.0%-165% increase in melanin content when compared to control. IS103 concentration at 250 μg/mL (160%) and 500 μg/mL (180%) showed similar results of standard positive controls IBMX (160%) and α-MSH (190%).

### Example 6.2: Tyrosine Activity Assay in B16F10 and NHEM Cell Lines

Tyrosinase activity was determined using L-DOPA as a substrate. Briefly, B16F10 and NHEM cells were seeded in a 6-well plate (2×10⁴ cells/well) and incubated for overnight. The cells were then treated with or without the test compounds for 46 hours and were washed twice with ice cold PBS and extracted by sonication in 100 μl of 0.1M Tris-HCl buffer (pH 7.2) containing 1% Nonidet P-40, 0.01% SDS, 100 μM phenyl methyl sulfonyl fluoride, and 1 μg/ml aprotinin and then centrifuged at 10,000 g for about 10 minutes at 4° C. The supernatants collected were used for enzyme assay and its protein content was estimated by method of Bradford using BSA as standard. Samples of cell extract supernatant were incubated in duplicate for 1 hour at 37° C. in 1 ml of 0.1M sodium phosphate buffer (pH 7.4) containing 0.1% L-DOPA. The absorbance was then monitored at 450 nm and compared with purified mushroom tyrosinase (Sigma). The standard curve was prepared limiting linearity within the range of experimental value.

### IS103 Increases Tyrosinase Activity

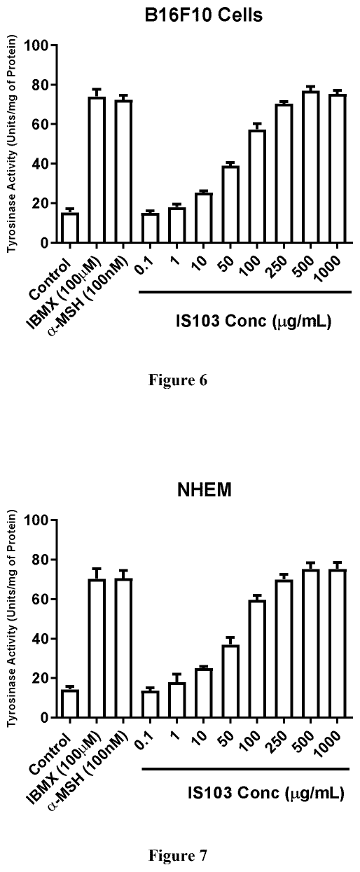

Since tyrosinase has been demonstrated to be the most critical molecule to regulate melanin synthesis, the inventor of present invention therefore analyzed whether IS103 could increase tyrosinase enzyme activity by tyrosinase activity assay using L-DOPA as a substrate. Consistent with the findings in study of effect on melanin synthesis, IS103 was able to enhance tyrosinase activity in a dose-dependent manner (Table 19 and FIGS. 6 and 7).

TABLE 19
TYROSINASE ACTIVITY IN B16F10 AND NHEM CELLS

| | B16F10 CELLS | | NHEM CELLS | |
|---|---|---|---|---|
| | | % increase in | | % increase in |
| | | melanin content | | melanin content |
| | Fold change in | compared to | Fold change in | compared to |
| Stimulants | melanin content | control | Melanin Content | control |
| Control (DMS0 0.1%) | 15.0 ± 2.50 | 0 | 14.33 ± 1.24 | 0 |
| IBMX (100 μM) | 74.0 ± 5.10 | 59 | 70.3 ± 4.20 | 56 |
| α- MSH (100 nM) | 72.3 ± 3.30 | 57.33 | 70.7 ± 3.10 | 56.33 |
| IS103 (0.1 μg/mL) | 15.0 ± 1.60 | 0 | 13.7 ± 1.24 | −0.67 |
| IS103 (1 μg/mL) | 18.0 ± 2.20 | 3 | 18.0 ± 3.30 | 3.67 |
| IS103 (10 μg/mL) | 25.3 ± 1.20 | 10.33 | 25.0 ± 0.80 | 10.67 |
| IS103 (50 μg/mL) | 39.0 ± 2.20 | 24 | 37.0 ± 3.00 | 22.67 |
| IS103 (100 μg/mL) | 57.3 ± 4.20 | 42.33 | 59.7 ± 1.90 | 45.33 |
| IS103 (250 μg/mL) | 70.3 ± 1.70 | 55.33 | 70.0 ± 2.20 | 55.67 |
| IS103 (500 μg/mL) | 77.0 ± 3.00 | 62 | 75.3 ± 2.50 | 61 |
| IS103 (1000 μg/mL) | 75.3 ± 2.50 | 60.33 | 75.3 ± 2.60 | 61 |

Results shown in the table 19 are Mean±SD obtained from triplicate experiments.

### CONCLUSIONS

B16F10 cells: IS103 (0.1 μg/mL-1000 μg/mL) demonstrated 0.0%-60.33% increase in Tyrosinase activity when compared to control in B16F10 cells. IS103 concentration at 250 μg/mL (55.33%) showed similar results of standard positive controls IBMX (59%) and α-MSH (57.33%).

NHEM cells: IS103 (0.1 μg/mL-1000 μg/mL) demonstrated 0.0%-60.33% increase in Tyrosinase activity when compared to control in NHEM cells. IS103 concentration at 250 μg/mL (55.67%) showed similar results of standard positive controls IBMX (56%) and α-MSH (56.33%).

Statistical analysis: All experiments were performed at least in triplicate. Values were represented as means±S.D. Statistical analyses were carried out using unpaired two-tailed Student's t tests and one way analysis of variance (ANOVA). Statistical significance was accepted at the level of P<0.05. All the statistical data analyses were analyzed by using Graph Pad Prism 5 software.

Conclusion: It was observed that IS103 increased melanogenesis without significantly affecting cell viability. The decapeptide increased in vitro tyrosinase activity and melanin content in a dose-dependent manner. The experimental results demonstrate increased in vitro tyrosinase activity without significant cytotoxicity to cells. Furthermore, the effect of IS103 on tyrosinase activity and melanin content was dose-dependent.

The experimental results indicate that the decapeptide induces melanogenesis in B16F10 cells primarily through increased tyrosinase expression and activity. The response of B16F10 and human primary melanocyte NHEM cell lines to IS103 indicates similarity to induction of melanin synthesis and tyrosinase activity. Therefore, the results provide basis of induced melanin formation. The results further establish a clear correlation between induction of melanin and increase in tyrosinase activity by the action of IS103.

These results demonstrate that IS103 induces pigmentation primarily through increased tyrosinase expression and activity. Hence, potent pigment inducing activity of IS103 is because of melanogenic property. The decapeptide is therefore a suitable candidate for treatment of hypopigmentation disorders and has a great potential for drug development.

### Example 7: Pre-Clinical Efficacy Evaluation of IS103 Dmso −0.1% Solution for the Treatment of Vitiligo in C57Bl/6 Male Mice

### 7.1: Vitiligo Model Induced by Repeated Application of Monobenzone

Briefly, C57BL/6 mice at 4 weeks of age were divided into six groups, 12 mice in each group. Animals had the dorsal region shaved, approximately 24 hours before. Monobenzone 40% was freshly prepared in non-ionic cream and daily applied (50 mL, for 65 days) to the dorsal region (2×2 cm²) on the same site near the tail. Once after the disease induction, after 65 days, the animals are randomized into the subgroups, 8 animals in each group. Creams were massaged until completely absorption using a spatula. In a different dorsal region (near the neck, 2×2 cm²), Melgain (0.1%), and IS103-DMSO (0.1%), (daily, once a day) was applied and massaged until completely absorption. Another group of animals receiving monobenzone was treated with vehicle. All treatments were carried out during 28 days, once after the induction of the disease. After, animals were euthanized, and 6 mm circles of ear tissue were collected. All tail and back skin were removed. Blood was collected and the serum was separated, and the samples were stored at −80° C. for further tests. Same dorsal skin and tail samples were collected and placed in 10% neutral buffered formalin to histological analysis. Samples also were used to evaluate depigmentation, and histological analyses (Hematoxylin and eosin staining), measurement of cytokines was done in the serum samples.

### 7.2: Histological Assessment of Skin Tissue

Ear and back samples were fixed in 10% neutral buffered formalin. Tissues were subsequently embedded in paraffin, sectioned to 5 mm and stained with hematoxylin-eosin. To evaluate number of stain nuclei field, histological sections stained with hematoxylin eosin of tail and back were photographed in increments of 200× and the photographs were analyzed with the ImageJ software version 1.47 (National Institute of Health, USA). Analyses were performed by counting cells per field. Ten fields from three distinct histological sections of each group were analyzed.

### 7.3: Measurement of IL-1B, IL-6, IL-10 and TNF-α Levels

Amounts of IL-1β, IL-6, IL-10 and TNF-α in the ear and dorsal tissue homogenates were quantified using ELISA kit (eBioscience, San Diego, USA) according to the manufacturer instructions. Levels of these cytokines in each supernatant were normalized to total protein content, which was determined using Bradford protein Laboratories, Hercules, CA, USA).

Depigmentation evaluation: The extent of depigmentation in the treated groups was analyzed by objective observation by two blinded observers. Each exposure location (monobenzone application site) and the extent of depigmentation (monobenzone non-application site) were examined and estimated as a depigmentation effect, which was the result of total depigmented sites observed in each animal/group.

Statistical analysis: The results were expressed as mean±S.E.M. Data were evaluated by one-way analysis of variance (ANOVA) followed by the Newman-Keuls post-hoc test. Po0.05 was considered as indicative of significance. The values were obtained using the Statistical software GraphPad Prism version 5.00, San Diego California, USA.

### Example 8: Evaluation of Effect of IS103-Dmso (0.1%) on Depigmentation Induced by Monobenzone (Vitiligo Model)

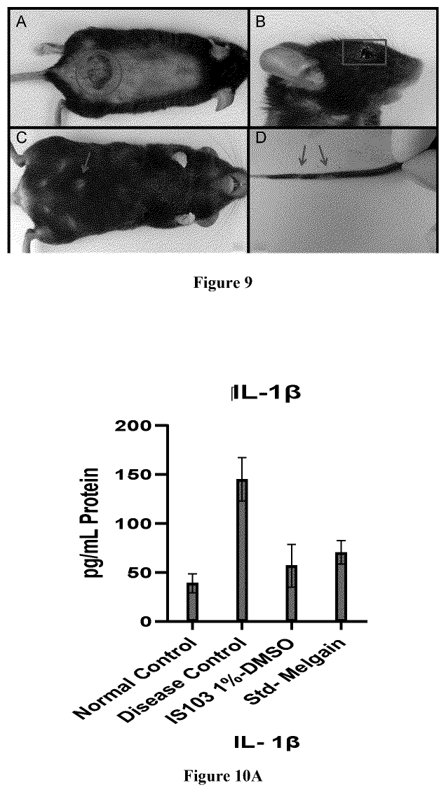

After topical application of monobenzone cream, small white patches appeared in the drug-exposed area and gradually expanded to surrounding areas (FIG. 9A). Distant white patches (depigmentation on the non-exposed sites) were gradually appearing on the eyelashes (FIG. 9B), abdomen (FIG. 9C), tail (FIG. 9D) and others sites on the back. On 65th day, depigmentation degrees were established for all treatments groups. All animals of control group (C) presented depigmentation on monobenzone application site (100% of animals tested). IS103-DMSO (0.1%) treatment by topical administration (0.1%) presented 90-95% of animals with depigmentation, respectively. The group receiving the reference drug shows 85% of animals with depigmentation on the application site. The percentage value refers only about the presence or not of depigmentation in the animal. Also, the depigmentation by monobenzone in non-application sites was evaluated and in the control group (monobenzone 40%) all the tested animals presented depigmentation.

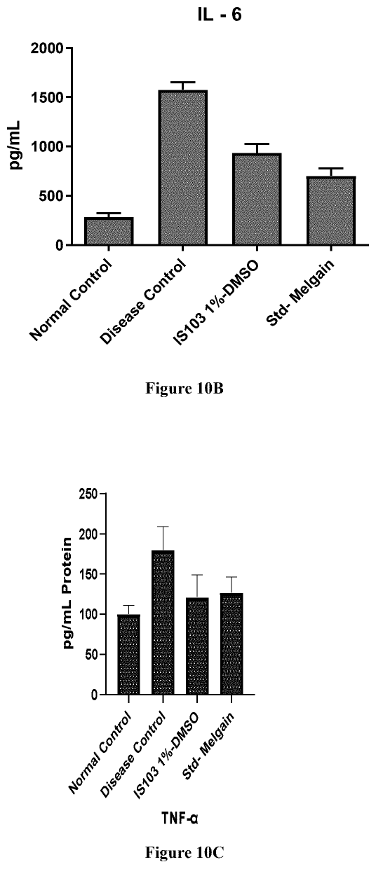

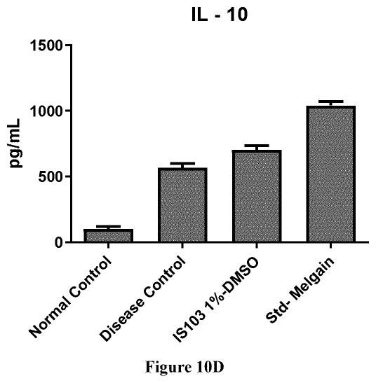

### Example 9: Estimation of Effect of IS103-Dmso (0.1%) on Cytokines in Serum Samples on Vitiligo Model (FIG. 10A-D)

Levels of tissue TNF-α were different between naïve and control group (FIG. 10C). Topical IS103-DMSO (0.1%) was capable to diminish TNF-α levels in 99.2712.1% (P<0.001).

The inventor of present invention investigated possible effects of IS103-DMSO (0.1%) in in vivo model, which mimic some features of pathogenesis of vitiligo. The treatment with IS103 DMSO solution was able to act on different parameters related to the etiopathogenesis of vitiligo, decreasing cellular infiltration, cytokine levels, and besides increasing melanin content and pigmentation. The results of various studies conducted confirm the efficacy of suitability of IS103-DMSO (0.1%) to treat vitiligo and white patches.

TABLE 20
DETAILS OF SOURCE OF CELL LINES AND ANIMAL
MODELS EMPLOYED IN THE PRESENT INVENTION

| Cell lines/mice models | Source | Country |
|---|---|---|
| Balb/c mice | Jeeva Lifesciences and housed at BITS-Pilani, Hyderabad | India |
| C57BL/6 mice | Jeeva Lifesciences and housed at BITS-Pilani, Hyderabad | India |
| B16F10 melanoma cell lines | ATCC ® CRL-6475 ™ | USA |
| NHEM melanoma cell lines | PromoCell with product code C-12403 | Germany |

TABLE 21
DETAILS OF SOURCE OF CELL LINES AND ANIMAL
MODELS EMPLOYED IN THE PRESENT INVENTION

| Chemical/Drug | Manufactured By | Batch No | Country |
|---|---|---|---|
| PEG | Himedia | A001A | India |
| Isopropyl alcohol | Standard Reagents | 67-63-0 | India |
| Sucrose | SD Fine chemicals ltd | 57-50-1 | India |
| | SDFCL | | |
| Penicillin/Streptomycin | Merck | 61751905001730 | USA |
| Fetal Bovine serum | Himedia | RM10685 | India |
| Veet Cream | Reckitt Benckiser India Pvt Ltd | NA | India |
| Dimethyl sulfoxide (DMSO) | Merck SpecialtiesPvt Ltd., | 67-68-5 | India |
| 3-isobutyl-1-methylxanthine (IBMX) | Sigma | I5879 | India |
| Melgain | Issar Pharmaceuticals pvt ltd., | NA | India |
| Monobenzone | Sigma -Aldrich | 103-16-2 | India |
| Mouse TNF-α ELISA kit | R & D Sys. | DY410-05 | USA |
| Mouse IL-6 ELISA kit | R & D Sys. | DY406-05 | USA |
| Mouse IL-10 ELISA kit | R & D Sys. | DY417-05 | USA |
| Mouse IL-1β ELISA kit | R & D Sys. | DY401-05 | USA |
| XTT- Cytotoxicity Assay Kit | Roche (sigma) | 11465015001-2KT | USA |
| Tyrosine Inhibition assay kit | Sigma | MAK257-2KT | India |
| αMSH | Sigma | M4135-5MG | India |
| L-DOPA | Sigma | D9628-100G | India |
| Mushroom Tyrosinase | Sigma | T3824-50KU | India |

### REFERENCES

1. Chinmoy Sarkar et al. (2006). Human placental protein/peptides stimulate melanin synthesis by enhancing tyrosinase gene expression. Molecular and Cellular Biochemistry 285:133-142.
2. Tsatmali M et al. (2002). Mealnocyte function and its control by melanocortin peptides. The Journal of Histochemistry Cytochemistry 50 (2): 125-133.
3. Thody A J (1993). Skin pigmentation and its regulation. In: G. C. Priestley, (ed). Molecular aspects of Dermatology. West Sussex, UK: John Willey & Sons Ltd., pp. 55-73.
4. Slominski A et al. (2004). Melanin pigmentation in mammalian skin and its hormonal regulation. Physiol Rev 84:1155-1228.
5. HSIU-CHIN HUANG et al. (2017). The lactoferricin B-derived peptide, LfB 17-34, induces melanogenesis in B16F10 cells. INTERNATIONAL JOURNAL OF MOLECULAR MEDICINE 39:595-602.
6. Tobin D J. (2011). The cell biology of human hair follicle pigmentation. Pigment Cell Melanoma Res 24:75-88.
7. Hsiao J J et al. (2014). The roles of microphthalmia-associated transcription factor and pigmentation in melanoma. Arch Biochem Biopsy 563:28-34.
8. Imokawa G et al. (1996). Siganlling mechanisms of endothelin induced mitogenesis and melanogenesis in human melanocytes. Biochem J 314:305-312.
9. Abdel-Malek Z et al. (1995). Mitogenic and melanogenic stimulation of normal human melanocytes by melanotropic peptides. Proc Natl Acad Sci USA 92:1789-1793.
10. Mcleod S D et al. (1995). Stimulation of tyrosinase in human melanocytes by pro-opiomelanocortin-derived peptides. J Endocrinol 146:439-447.
11. Hunt G et al. (1994). ACTH stimulates melanogenesis in cultured human melanocytes. J Endocrinol 140: R1-R3.
12. Hunt G et al. (1994). α-Melanocyte stimulating hormone and its analogue Nle4Dphe7α-MSH affect morphology, tyrosinase activity and melanogenesis in cultured human melanocytes. J Cell Sci 107:205-211.
13. Pawlek J M (1976). Factors regulating growth and pigmentation of melanoma cells. J Invest Dermatol 66:201-209.
14. Seigrist W et al. (1986). In situ melanin assay for MSH using mouse B16 melanoma cells in culture. Anal Biochem 159:191.
15. Kauser et al. (2003). Regulation of human epidermal biology by beta-endorphin. J Invest Dermatol 120:1073-1080.
16. Dessinioti C et al. (2009). A review of genetic disorders of hypopigmentation: Lessons learned from the biology of melanocytes. Exp Dermatol 18:741-749.
17. Panhard et al. (2012). Greying of the human hair: A worldwide survey, revisiting the ‘50’ rule of thumb. Br J Dermatol 167:865-873.
18. Ezzedine et al. (2015). Vitiligo. Lancet 386:74-84.
19. Falabella et al. (2009). Update on skin repigmentation therapies in vitiligo. Pigment Cell Melanoma Res 22:42-65.
20. World Health Organisation—WHO Technical Report Series No. 953. 2009. Annex-2. Stability testing of active Pharmaceutical ingredients and finished Pharmaceutical products.
21. Schedule Y, http://cdsco.nic.in/html/schedule-y % 20 (amended %20version-2005) % 20original.html
22. International Conference on Harmonization (2003) Q1A (R2): Stability testing of new drug substances and products (second revision).
23. International Conference on Harmonization (2003) Q1E: Evaluation of stability data.
24. OECD (2015), Test No. 404: Acute Dermal Irritation/Corrosion, OECD Guidelines for the Testing of Chemicals, Section 4, OECD Publishing, Paris, https://doi.org/10.1787/9789264242678-en.

Although the subject matter has been described herein with reference to certain preferred embodiments thereof, other embodiments are possible. For illustrative purpose, the formulation of invention has been tested for treatment, preventing and/or ameliorating one or more symptoms of hypo-pigmentary disorders. However, those skilled in the art would appreciate that scope of the invention would extend to other pigmentary disorders. The invention may be embodied in other specific forms without departing from the spirit or essential characteristics thereof. The foregoing embodiments are therefore to be considered in all respects illustrative rather than limiting the invention described herein. It will be obvious to those skilled in the art to make various changes, modifications and alterations to the invention described herein. To the extent that these various changes, modifications and alteration do not depart from the scope of the present invention, they are intended to be encompassed therein. In addition, where this application has listed the steps of a method or procedure in a specific order, it may be possible, or even expedient in certain circumstances, to change the order in which some steps are performed, and it is intended that the particular steps of the method or procedure claims set forth herein below not be construed as being order-specific unless such order specificity is expressly stated in the claim. Scope of the invention is thus indicated by the appended claims rather than by the foregoing description, and all changes that come within the meaning and range of equivalency of the claims are intended to be embraced therein.

1. A pharmaceutical formulation comprising 0.01 to 1% w/v of a peptide of SEQ. ID NO 1 or variant thereof and one or more suitable pharmaceutically acceptable excipients for treating, preventing and/or ameliorating one or more symptoms of pigmentary disorders.

2. The formulation as claimed in claim 1, wherein said one or more suitable pharmaceutically acceptable excipients are selected from the group consisting of suitable carriers, diluents, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants.

3. The formulation as claimed in claim 1, wherein said peptide variant is at least 80%, at least 85%, at least 90%, at least 95%, at least 97%, at least 99% identical to the SEQ. ID NO 1.

4. The formulation as claimed in claim 1, wherein said formulation is suitable for topical mode of administration and said formulation is in form of a gel, ointment, creams, lotion, solution and foams.

5. The formulation as claimed in claim 1, wherein the pigmentary disorders are hypopigmentation disorders from the group vitiligo, pityriasis alba, tinea versicolor, albinism, piebaldism, tuberous sclerosis, hypo melanosis of Ito.

6. The formulation as claimed in claim 1, for prevention and/or delay of progression of vitiligo or for reversal of depigmentation and/or increasing melanin of patients suffering from vitiligo.

7. The formulation as claimed in claim 1, wherein said one or more symptoms of pigmentary disorders are patchy loss of skin color, premature whitening or graying of hair, loss of color in tissues.

8. A method of preparing pharmaceutical formulation for treating, preventing and/or ameliorating one or more symptoms of pigmentary disorders comprising the steps:
a) adding required quantity of water and decapeptide of SEQ. ID NO 1 or variant thereof in a container and stirring at suitable speed by suitable means until clear solution is obtained;
b) adding required quantity of sucrose in a separate container and stirring at suitable speed by suitable means until completely dissolved;
c) adding sucrose solution obtained in step ‘b’ to the clear solution obtained in step ‘a’;
d) adding required quantity of pharmaceutically acceptable excipients to the clear solution obtained in step ‘a’ under continuous stirring until a clear homogenous solution of the pharmaceutical formulation is obtained.

9. The method as claimed in claim 8, further comprising packaging of said homogenous solution in suitable containers.

10. The method as claimed in claim 8, optionally comprising sterilizing the pharmaceutical formulation by suitable sterilization methods before or after packaging of said pharmaceutical formulation in suitable containers.

11. The method as claimed in claim 8, wherein said suitable speed in step ‘a’ is 150 rpm.

12. The method as claimed in claim 8, wherein said pharmaceutically acceptable excipients in step ‘d’ comprises isopropyl alcohol, dimethyl sulfoxide and propylene glycol.

13. A method of treating, preventing and ameliorating one or more symptoms of pigmentary disorders in a subject in need thereof, said method comprising administering a therapeutically effective amount of a formulation comprising a therapeutically effective amount of peptide of SEQ. ID NO 1 or variant thereof wherein the formulation enhances melanin synthesis and/or suppresses autoimmune targeting of melanocytes and/or inhibits tyrosinase activity and/or reduces levels of cytokines.

14. The method as claimed in claim 13, wherein said therapeutically effective amount of peptide of SEQ. ID NO 1 or variant thereof in said formulation is 0.01 μg/ml to 10,000 μg/ml.

15. The method as claimed in claim 14, wherein said therapeutically effective amount of peptide of SEQ. ID NO 1 or variant thereof in said formulation is preferably 0.1 μg/ml to 1000 μg/ml.

16. The method as claimed in claim 13, wherein said formulation is administered to the subject through topical mode of administration.

17. (canceled)

18. (canceled)

19. The method claim 8, wherein the method further comprises manufacturing decapeptide IS103 of SEQ. ID NO 1 or variant thereof according to the steps:
a) synthesizing said decapeptide by coupling one amino acid at a time, starting from C-terminus amino acid which is attached to a solid resin via a linker group;
b) controlling coupling of step a) by varying de-protection time and reagents, wherein de-protection is performed twice;
c) drying and weighing peptide resin obtained after coupling last amino acid;
d) cleaving resin-bound peptide off said resin by trifluoroacetic acid to obtain crude peptide;
e) optionally processing crude peptide obtained in step d) by reverse phase chromatography and ion exchange to obtain solution of purified peptide;
f) optionally lyophilizing said solution of purified peptide for removal of residual solvents.


SEQUENCE LISTING (1 sequence)

SEQ ID NO: 1 - protein, 10 aa, synthetic construct
  source 1..10: note = IS103
YRSRKYSSWY